(19) United States
(12) Patent Application Publication
(10) Pub. No.: US 2021/0052409 A1
(43) Pub. Date: Feb. 25, 2021

(54) Articulating Clamshell for Ankle Foot Orthosis and Method of Use Thereof

(71) Applicant: Sanjay Upadhyaya, Spring, TX (US)

(72) Inventor: Sanjay Upadhyaya, Spring, TX (US)

(21) Appl. No.: 16/544,898

(22) Filed: Aug. 19, 2019

(51) Int. Cl. A61F 5/01

(52) U.S. Cl. CPC A61F 5/0127; A61F 2005/0158

(57) ABSTRACT

An articulating clamshell for ankle foot orthosis and method of use thereof is described herein. A proximal articulation clamshell (PACS) system can comprise a fixed shell, a movable shell, and a fastening device. The fixed shell can comprise a fixed shell proximal end and a fixed shell distal end. The fixed shell can form-fit with a portion of a lower leg. The movable shell can comprise a movable shell proximal end and a movable shell distal end. The moveable shell can form-fit with a second portion of the lower leg. The movable shell proximal end can connect with the fixed shell proximal end wherein the movable shell can articulate in relation to the fixed shell. The movable shell can be capable of being in an open position and a closed position. The fastening device can secure the movable shell in the closed position.

### BACKGROUND

This disclosure relates to an articulating clamshell for ankle foot orthosis and method of use thereof. For years, ankle foot orthosis (AFO) that is worn on the lower leg is used in orthotics to provide total contact on the shin, and to restrict, assist, limit or control ankle motion. Orthotists can include use of AFO in order to treat and improve various conditions of the feet caused by medical conditions such as foot and ankle pain, foot drop, or deformities caused by stroke, cerebral palsy, multiple sclerosis, or injury. A typical clamshell for AFO usually comprises an anterior shell and a posterior shell, which can be held together on the shin by using two straps. The first strap can be placed at the proximal part of the shell while the second strap can be placed at the distal part of the shell. The two straps can secure the anterior shell and the posterior shells to the lower leg. Once worn, an AFO can stabilize the tibia in both the frontal and sagittal plane, which can allow a user to move his leg in these planes effectively. However, individuals such as hemiplegic patients, who can only control one of their hands, might find it difficult to put on or take of an AFO by themselves using the standard clamshell, but instead need assistance from family members, nurses or therapists. Such repetitive tasks can be frustrating and can cause emotional distress and compliance issues, hindering their ability to be self-dependent, since it can make them feel incapacitated. Additionally, straps on conventional clamshells can be placed at the cuff and ankle, which can apply pressure near the origin and insertion of the muscles inhibit muscle contraction. Lastly, use of multiple straps directly over the skin can cause skin break down and sores caused by excessive pressures resulting from the dynamic load applied by the straps that uses a three-point force system. As such it would be useful to have an improved clamshell for ankle foot orthosis.

### SUMMARY

An articulating clamshell for ankle foot orthosis and method of use thereof is described herein. A proximal articulation clamshell (PACS) system can comprise a fixed shell, a movable shell, and a fastening device. The fixed shell can comprise a fixed shell proximal end and a fixed shell distal end. The fixed shell can form-fit with a portion of a lower leg, and the fixed shell can extend approximately half way around the lower leg. The movable shell can comprise a movable shell proximal end and a movable shell distal end. The moveable shell can form-fit with a second portion of the lower leg. The movable shell can extend approximately half way around the lower leg. The movable shell proximal end can connect with the fixed shell proximal end wherein the movable shell can articulate in relation to the fixed shell. The movable shell can be capable of being in an open position and a closed position. In the open position, the fixed shell and the movable shell can form an opening that can be capable of receiving the lower leg. In the closed position, the fixed shell and the movable shell together can extend substantially around the lower leg. The fastening device can be at the moveable shell distal end that can secure the movable shell in the closed position.

A method of wearing and removing an improved proximal articulation clamshell (PACS) system comprising the step of putting a PACS system into an open position. The PACS can comprise a fixed shell, a movable shell, and a fastening device. The fixed shell can comprise a fixed shell proximal end and a fixed shell distal end. The fixed shell can form-fit with a portion of a lower leg, and the fixed shell can extend approximately half way around the lower leg. The movable shell can comprise a movable shell proximal end and a movable shell distal end. The moveable shell can form-fit with a second portion of the lower leg. The movable shell can extend approximately half way around the lower leg. The movable shell proximal end can connect with the fixed shell proximal end wherein the movable shell can articulate in relation to the fixed shell. The movable shell can be capable of being in the open position and a closed position. In the open position, the fixed shell and the movable shell can form an opening that can be capable of receiving the lower leg. In the closed position, the fixed shell and the movable shell together can extend substantially around the lower leg. The fastening device can be at the moveable shell distal end that can secure the movable shell in the closed position. The method can also comprise the steps of placing the lower leg within the PACS system, putting the PACS system into closed position, and securing the PACS system into closed position using the fastening device.

### BRIEF DESCRIPTION OF THE DRAWINGS

FIG. 1A illustrates an embodiment of anterior-opening proximal articulation clamshell (PACS) system attached to a right shoe through a vertical support and footplate.

FIG. 1B illustrates and embodiment of anterior opening PACS without attachment.

FIG. 2A illustrates an embodiment of PACS system with an anterior opening.

FIG. 2B illustrates an embodiment of PACS system with an anterior opening with a combined vertical support and fixed shell.

FIG. 3 illustrates a top view of PACS system with an anterior opening.

FIG. 4 illustrates a back view of PACS system with an anterior opening.

FIG. 5 illustrates an interior surface of PACS system comprising a cushion.

FIG. 6 illustrates PACS system with an anterior opening worn on a leg.

FIG. 7 illustrates an embodiment of PACS system that can comprise a lateral opening.

FIG. 8 illustrates an embodiment of PACS system with a lateral opening in a closed position

FIG. 9 illustrates PACS system with a lateral opening worn on a leg.

FIG. 10 illustrates another embodiment of PACS system comprising a medial opening.

FIG. 11 illustrates PACS system with a medial opening worn on a leg.

### DETAILED DESCRIPTION

Described herein is an articulating clamshell for ankle foot orthosis and method of use thereof. The following description is presented to enable any person skilled in the art to make and use the invention as claimed and is provided in the context of the particular examples discussed below, variations of which will be readily apparent to those skilled in the art. In the interest of clarity, not all features of an actual implementation are described in this specification. It will be appreciated that in the development of any such actual implementation (as in any development project), design decisions must be made to achieve the designers' specific goals (e.g., compliance with system- and business-related constraints), and that these goals will vary from one implementation to another. It will also be appreciated that such development effort might be complex and time-consuming, but would nevertheless be a routine undertaking for those of ordinary skill in the field of the appropriate art having the benefit of this disclosure. Accordingly, the claims appended hereto are not intended to be limited by the disclosed embodiments, but are to be accorded their widest scope consistent with the principles and features disclosed herein.

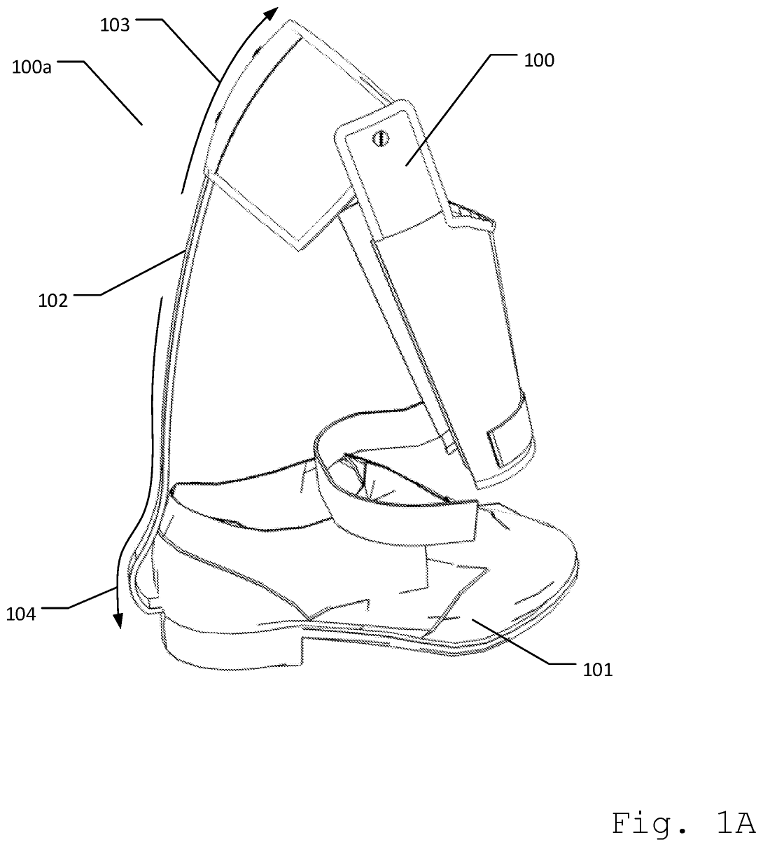

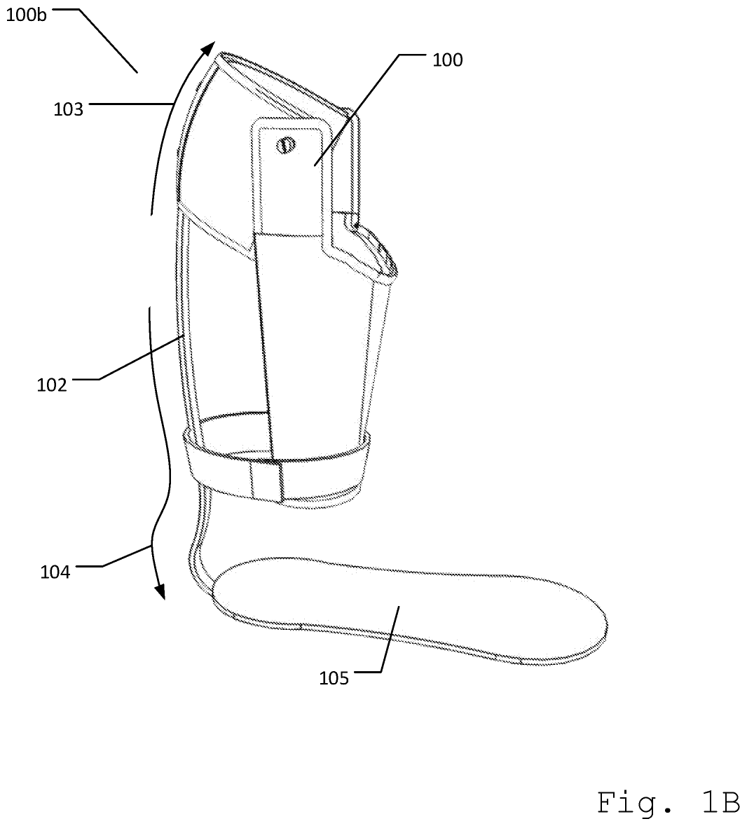

FIG. 1A illustrates an embodiment of proximal articulation clamshell (PACS) system 100 connected to a right shoe 101 through a vertical support 102. Individuals who suffer from different medical conditions such as stroke, and cerebral palsy, which can allow their ankle to roll while walking, can use PACS system 100 for ankle foot orthosis. As such, PACS system 100 can provide support to weakened ankle joint or an ankle joint that has lost control. Moreover, PACS system 100 can ensure that a foot is kept in a neutral position. In one embodiment, PACS system 100 can be capable of being in an open position 100a, and a closed position 100b. In such embodiment, a movable support can be articulated into open position 100a and closed position 100b. Vertical support 102 can secure PACS system 100 to shoe 101 in a stable upright position. An example of vertical support 102 can include but are not limited to carbon fiber strut and footplate. Vertical support 102 can comprise a vertical support proximal end 103, and a vertical support distal end 104. In one embodiment, a fixed shell of PACS system 100 can mount vertical support proximal end 103 while vertical support distal end 104 can mount shoe 101. In such embodiment, vertical support distal end 104 can affix at the rear portion of shoe 101 by sliding within the soles of the shoe, as shown in FIG. 1. In another embodiment, vertical support 102 can have an L-shape form wherein the horizontal distal end portion of vertical support 102 can be inserted and can be used to replace an insole of shoe 101, which will be discussed and shown below.

FIG. 1B illustrates another embodiment of vertical support 102 comprising a footplate 105. In this embodiment, vertical support distal end 104 can comprise footplate 105. Footplate 105 can be the horizontal portion of vertical support 102. In one embodiment, footplate 105 can be inserted and can be used to replace an insole of shoe 101. In such embodiment, footplate 105 can have the same shape as an insole of a shoe. Such embodiment can allow a user to change shoes easier.

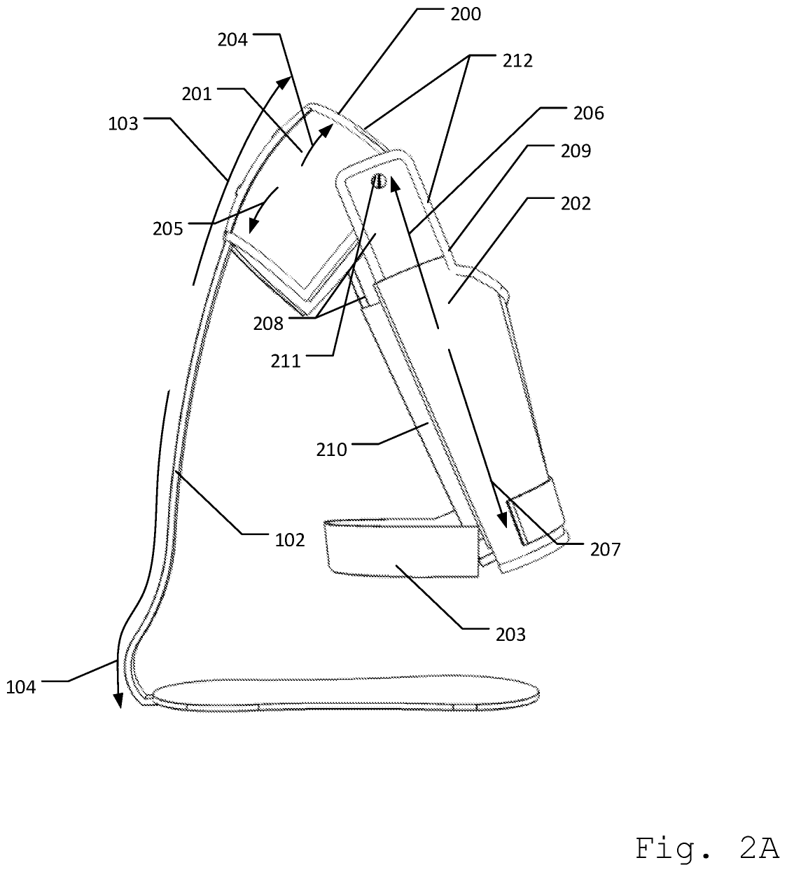

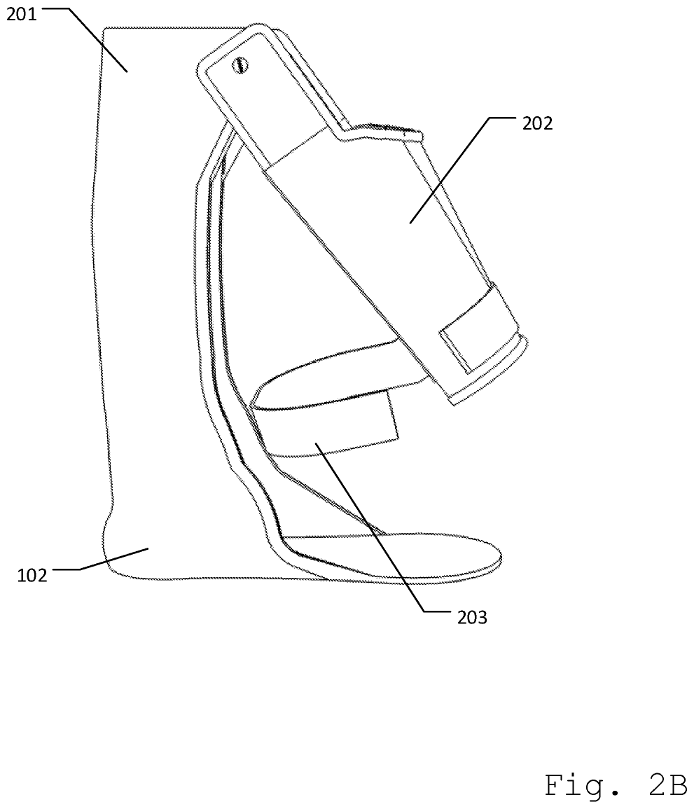

FIG. 2A illustrates an embodiment of PACS system 100 with an anterior opening 212. In one embodiment, PACS system 100 can comprise a leg access 200, a fixed shell 201, a movable shell 202, and a fastening device 203. Leg access 200 can be an opening at the proximal end of PACS system 100 capable of receiving the lower leg. Fixed shell 201 can provide a fixed support at a portion of the lower legs while movable shell 202 can provide support at a second portion of the lower legs. Fixed shell 201 can comprise a fixed shell proximal end 204 and a fixed shell proximal end 205. In one embodiment, fixed shell 201 can mount at vertical support proximal end 103. In such embodiment, vertical support 102 can provide an additional support to the lower legs. Movable shell 202 can comprise a movable shell proximal end 206 and a movable shell distal end 207. In one embodiment, movable shell proximal end 206 can comprise a pair of prongs 208. Prongs 208 can form a U-shape gap 209 that can provide an extended opening for leg access 200. In one embodiment, leg access 200 together with gap 209 can provide an enlarge opening capable of receiving the lower leg. In such embodiment, leg access 200 can be an anterior opening, a lateral opening, or a medial opening.

In one embodiment, fixed shell 201 and movable shell 202 can each be a C-shaped clamshell, which when attached together can form an interior surface 210. In such embodiment, movable shell proximal end 206 can connect with fixed shell proximal end 204 wherein movable shell 202 can articulate in relation to fixed shell 201. As such, prongs 208 can articulate with fixed shell proximal end 204 with a pair of hinges 211. In such embodiment, each hinge 211 can be placed on medial and lateral sides proximally. Such structure can allow movable shell 202 to swing to and/or from fixed shell 201 in the direction of the sagittal plane creating anterior opening 212. Furthermore, swinging movable shell 202 forward and away from fixed shell 201 in the direction of a sagittal plane can enlarge anterior opening 212. Thus, movable shell 202 can be capable of being in an open position 100a, and a closed position 100b. In open position 100a, movable shell 202 can be articulated away from fixed shell 201. In such position, leg access 200 aligned with gap 209 can form a larger opening capable of receiving the lower leg. In closed position 100b, movable shell 202 can be articulated towards fixed shell 201. As such, in closed position 100b, movable shell 202 together with fixed shell 201 can extend substantially around the lower leg. Such structure can allow the user to adjust pressure

Further in one embodiment, movable shell 202 can comprise fastening device 203. In one embodiment, fastening device 203 can be a strap. In one embodiment, the strap can be attached to movable shell distal end 207. In this embodiment, fastening device 203 can secure the distal end of PACS system 100 and vertical support 102 to the legs of the user.

FIG. 2B illustrates an embodiment of PACS system 100 with anterior opening 212 with a combined vertical support 102 and fixed shell 201. In one embodiment, vertical support 102 and fixed shell can combine. Fixed shell 201 can extend downward to footplate 105. A user can put foot in PACS system 100, and then put the foot and PACS system 100 in a shoe.

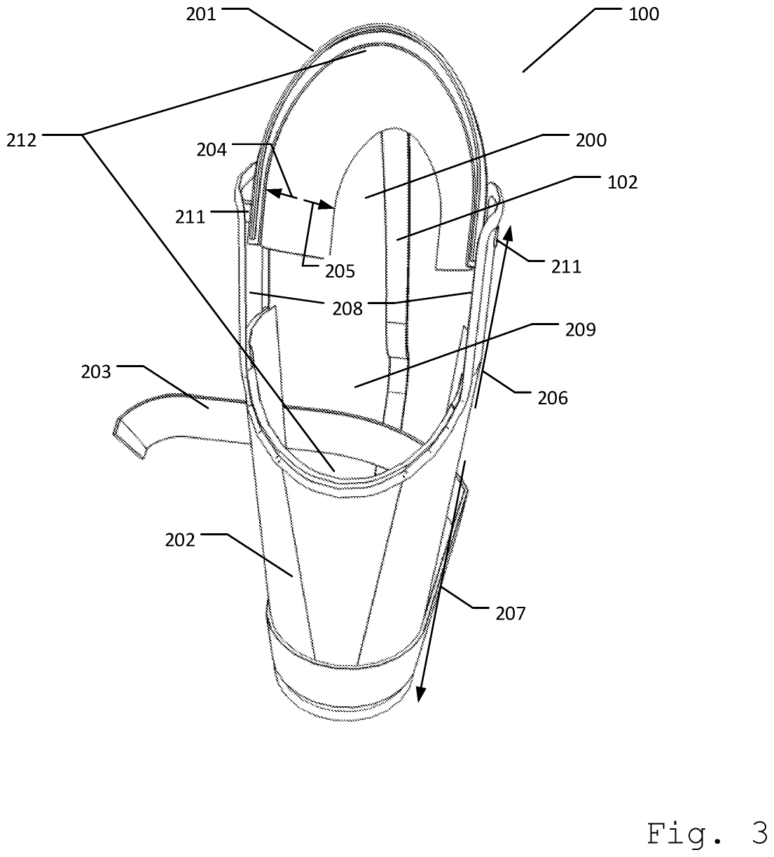

FIG. 3 illustrates a front view of PACS system 100 with anterior opening 212. In this embodiment, the proximal half of movable shell 202 can be trimmed out thus forming a U-shape in order to facilitate the receiving of the affected lower leg. In this embodiment, fixed shell 201 can be positioned at the posterior section of PACS system 100 while movable shell 202 can be positioned at the anterior section of PACS system 100. In such embodiment, fixed shell 201 can form-fit the posterior portion of the lower legs, while movable shell 202 can form-fit the anterior portion of the lower legs. As such, fixed shell 201 can extend approximately half way around the posterior portion of the lower leg while movable shell 202 can extend approximately half way around the anterior portion of the lower leg. Furthermore, in this embodiment, fixed shell 201 can be shorter than movable shell 202. In such embodiment, fixed shell 201 can provide less contact to the surface of the leg, thus can allow better fluid circulation on the leg. Furthermore, PACS system 100 with anterior opening 212 when attached to vertical support 102 can be used as a night splint. As such, PACS system 100 can reduce planter flexion contracture on the lower legs. Planter flexion contracture can be reduced by adding rings to proximal hinges 209 and by adding straps on footplate 105.

Moreover, such structure of PACS system 100 can be breathable, lighter and comfortable to the user. Fastening device 203 can secure PACS system 100 to the leg and can lock movable shell 202 in place, preventing movable shell 202 from swinging open when PACS system 100 is used for walking or standing. In an embodiment wherein fastening device 203 can be a strap, fastening device 203 can comprise a hook and loop fastener. Alternatively device 203 can be any other fastener known in the art such as a strap and buckle clasp as with inline skates or a cinch strap. In another embodiment, PACS system 100 can have two pieces that mate and lock together with some means of mechanical actuated release to allow opening of PACS system 100.

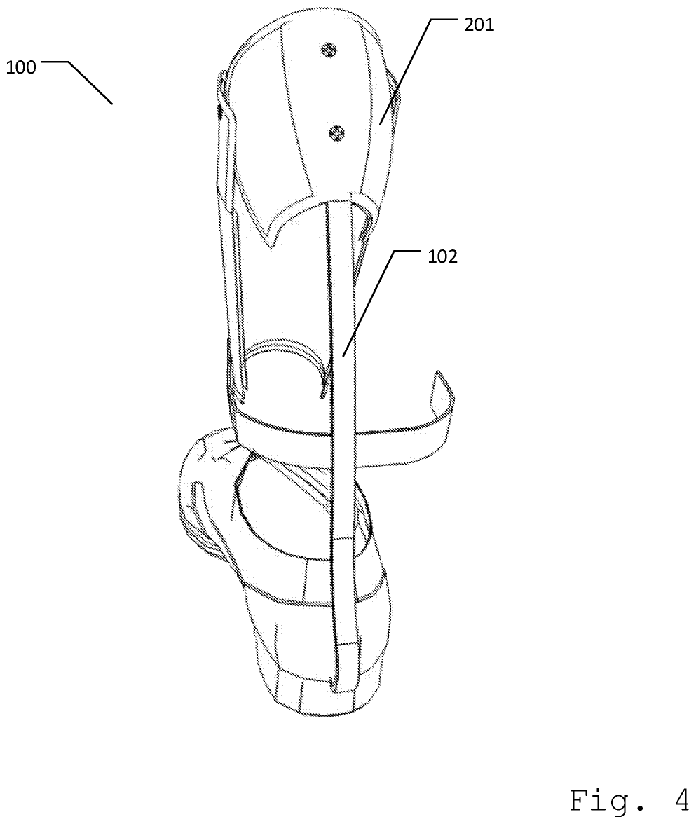

FIG. 4 illustrates a back view of orthotic brace 100 comprising anterior opening 212. In one embodiment, fixed shell 201 can permanently attach to vertical support 102 through methods that can include but is not limited to fastening, sewing, cementing, molding, or adhesion. In other embodiments, fixed shell 201 can be attachable and detachable to vertical support 102 through a fastener.

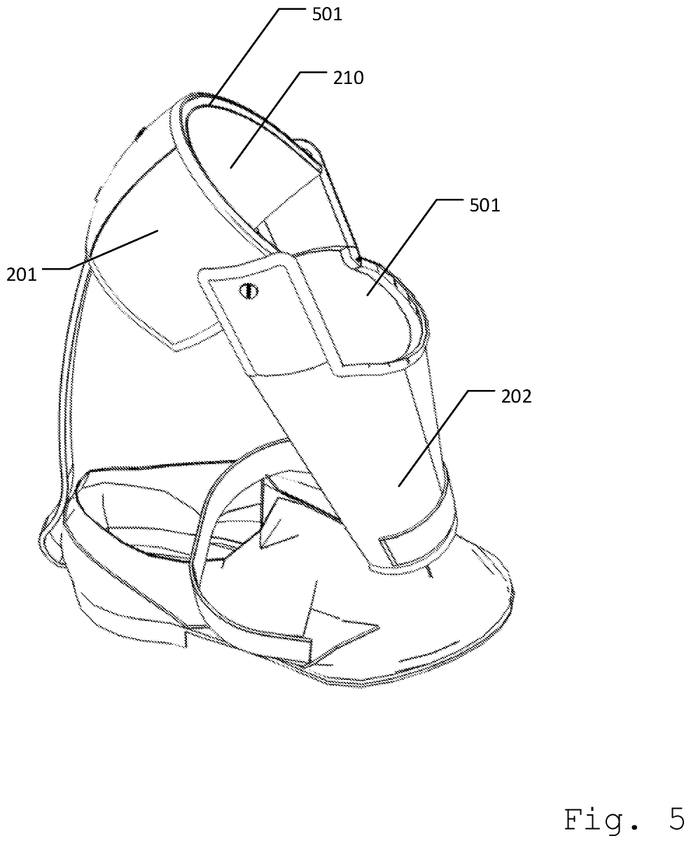

FIG. 5 illustrates interior surface 210 of PACS system 100 comprising a cushion 501. Cushion 501 can be made of soft material such as a fabric to provide comfortable support to the individual wearing PACS system 100. In one embodiment, cushion 501 can cover the entire interior surface 205 of fixed shell 201 and movable shell 202. In one embodiment, cushion 501 can permanently attach to cushion 501 through method that can include but is not limited to adhesion or fastening. In another embodiment, cushion 501 can be removable from PACS system 100. In this embodiment, cushion 501 can attach within interior surface 210 through a fastener such as a hook and loop fattener, a snap, or a button. In such embodiment, cushion 501 can be washed and/or be replaced with a new cushion.

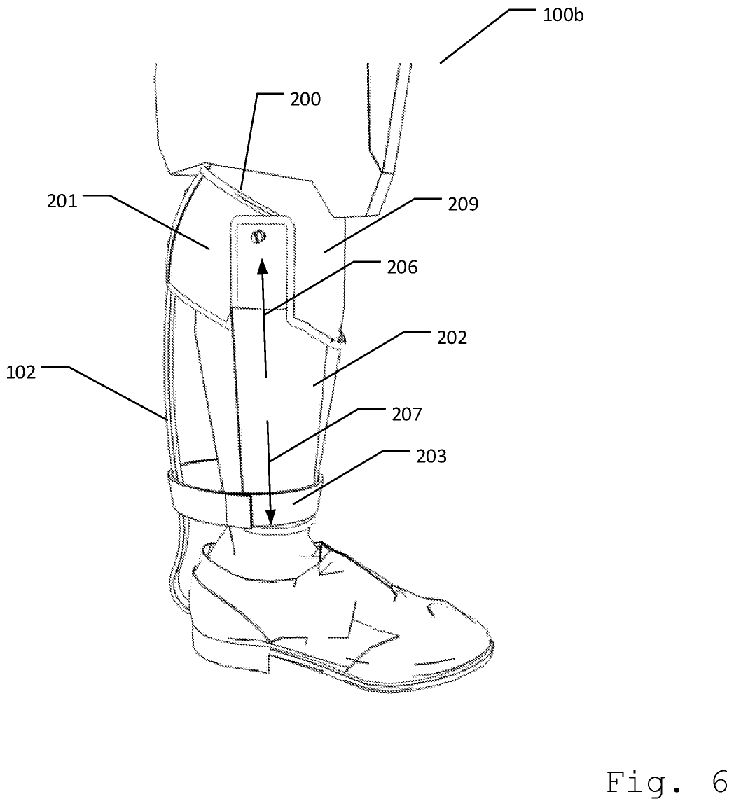

FIG. 6 illustrates PACS system 100 with anterior opening 212 worn on the legs. In this embodiment, PACS system 100 can be used for the management of foot drop or equinus foot. Foot drop or equinus foot can be a condition in which muscle weakness or paralysis can make it difficult to lift the forefoot. In such embodiment, gap 209 at movable shell proximal end 206 can allow a larger leg access 200, which can facilitate the insertion of the foot into PACS system 100. In an example scenario, the user can put PACS system 100 into open position 100a by swinging movable shell distal end 207 away from fixed shell 201. Next, the user can slip his foot into leg access 200. Once the foot is in place the user can put PACS system 100 into close position 100b by swinging movable shell distal end 207 towards the anterior portion of the leg. In an embodiment wherein fastening device 203 can be a strap, the user can wrap fastening device 203 around movable shell 202, fixed shell 201, and vertical support 102 that can lock the distal tibia in place. Thus, in this scenario the user can perform the tasks of opening, wearing, and fastening PACS system 100 on his shin, all by himself. Further in this embodiment, to remove PACS system 100 the user can also use one hand to unlock fastening device 203, which can loosen movable shell 202, and can allow user to remove PACS system 100 from the leg.

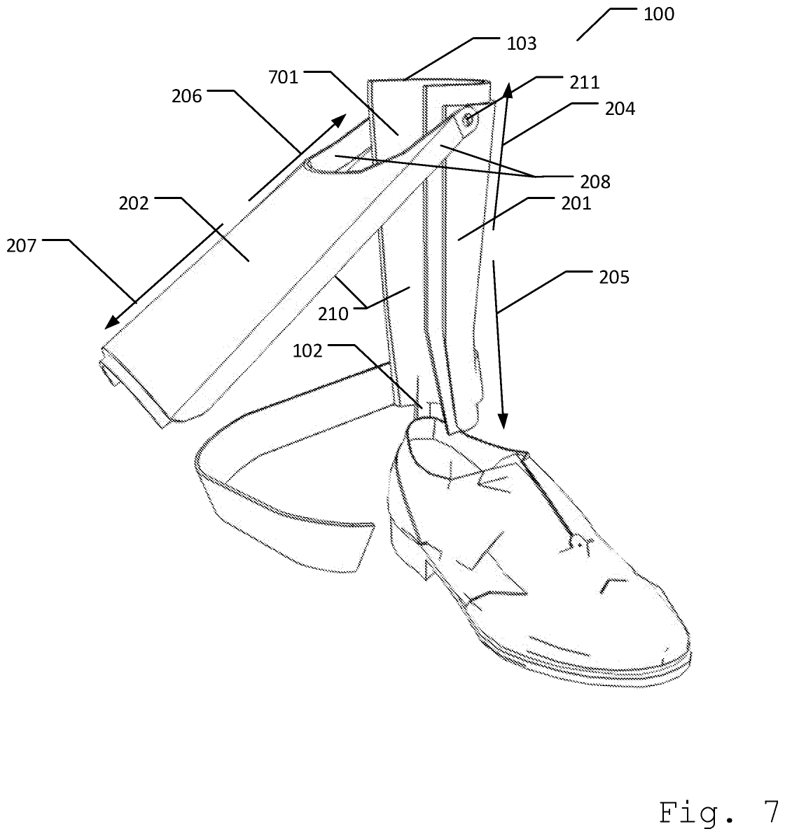

FIG. 7 illustrates an embodiment of PACS system 100 that can comprise a lateral opening 701. This embodiment can be used for the management of varus instability or deformity foot. Varus deformity can imply an angulation toward the midline of the distal segment of bone or joint, which can be caused by muscle imbalance. In such embodiment, fixed shell 201 can be placed at the medial section of the lower leg while movable shell 202 can be placed at the lateral section of the lower leg. As such, the user can open PACS system 100 by swinging movable shell distal end 207 away from fixed shell 201 and towards the lateral section, thereby enlarging lateral opening 701. In such embodiment, interior surface 210 of movable shell 202 can be facing the medial plane, while interior surface 210 of fixed shell 201 can be facing the lateral plane. In one embodiment, the medial half of fixed shell 201 can attach to vertical support proximal end 103. In such embodiment, hinges 211 can articulate fixed shell 201 and movable shell 202 proximally, such that each hinge 211 can attach prongs 208 to fixed shell proximal end 204 at the anterior and posterior portion of PACS system 100. In this embodiment, fixed shell 201 can support the medial portion of the lower leg while movable shell 202 can support the lateral portion of the lower leg. Such can apply a medially directed force on the distal tibia to stabilize an ankle in the frontal plane by shifting the ground reaction force lateral to the ankle joint.

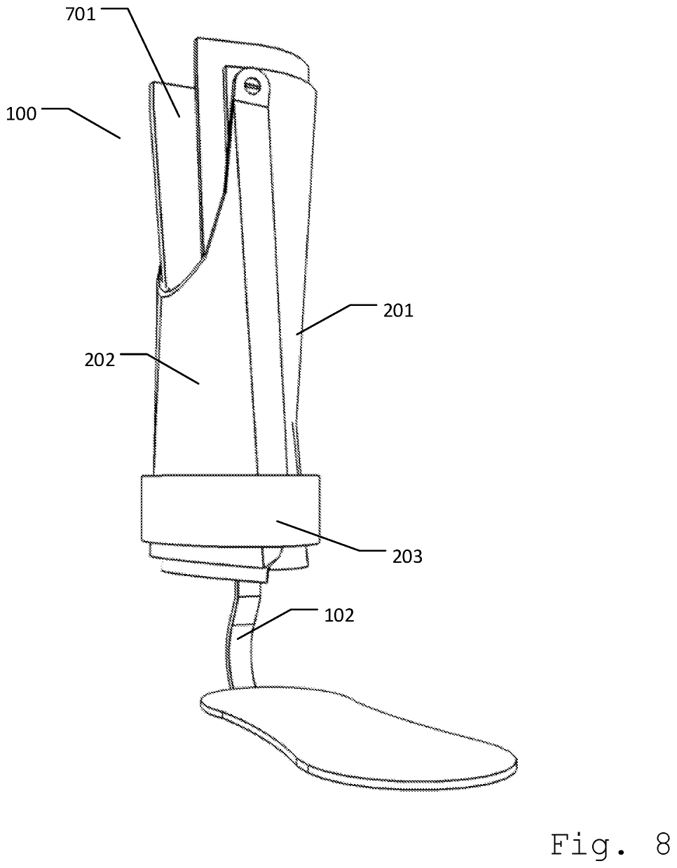

FIG. 8 illustrates an embodiment of PACS system 100 with lateral opening 701 in closed position 100b. In this embodiment fixed shell 201 and movable shell 202 can substantially be the same length. Such embodiment can allow PACS system 100 to cover a large surface of the lower leg and can provide support to the areas between the knee and the ankle. In this embodiment, the posterior portion of the lower leg can be substantially covered with PACS system 100. In one embodiment, vertical support 102 can be attached to fixed shell 201. In another embodiment, vertical support 102 can be embedded within fixed shell 201. In such embodiment, vertical support 102 and fixed shell 201 can be a single device.

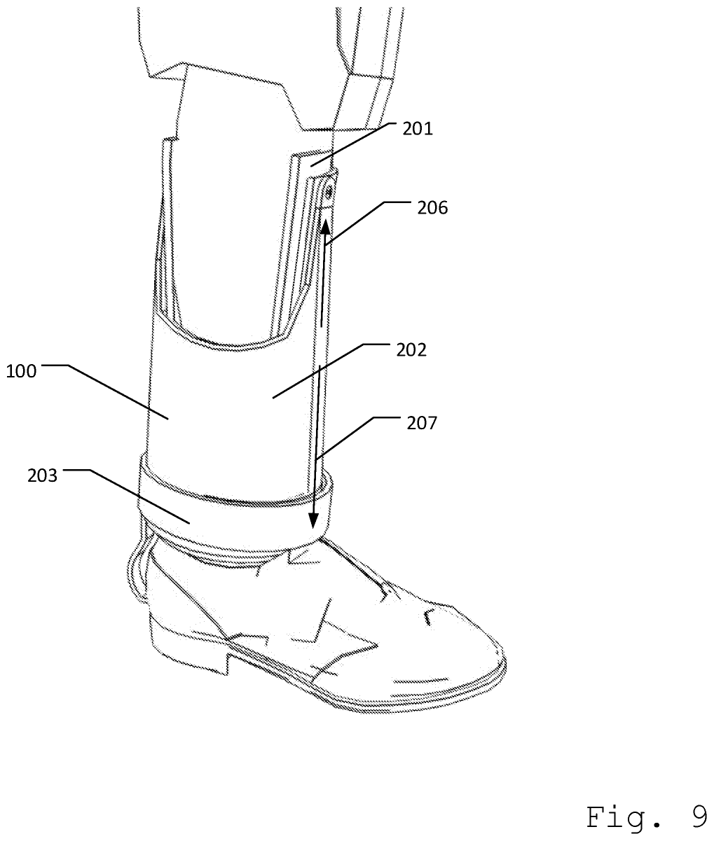

FIG. 9 illustrates PACS system 100 with lateral opening 701 worn on the leg. In this embodiment, the user can put PACS system 100 into open position 100a by swinging movable shell distal end 207 laterally away from fixed shell 201. This can create a wider lateral opening 701 that can facilitate the insertion of the lower leg within PACS system 100. Once the lower leg is in place, the user can put PACS system 100 into close position 100b by pulling movable shell distal end 207 towards the lateral portion of the lower leg. Then, fastening device 203 can be used to wrap PACS system 100 with the lower leg.

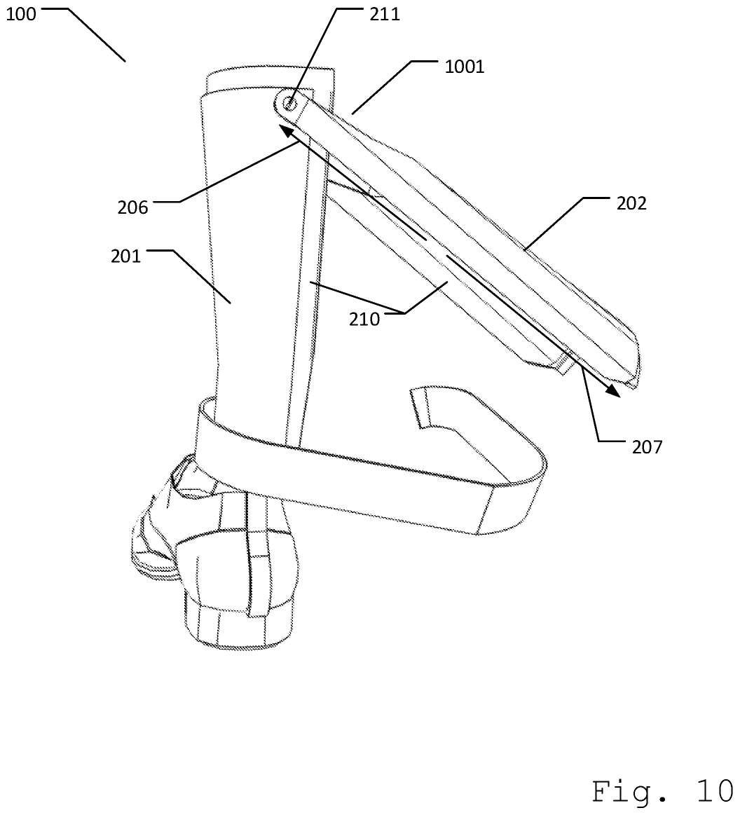

FIG. 10 illustrates another embodiment of PACS system 100 comprising medial opening 1001. This embodiment can be used for the management of valgus foot. In this embodiment, fixed shell 201 can be positioned at the lateral section of the legs while movable shell 202 can be positioned at the medial section of the legs. In such embodiment, the user can open PACS system 100 by swinging movable shell distal end 207 towards the medial direction and away from fixed shell 201, which can enlarge medial opening 1001. In this embodiment, movable shell 202 can overlap portions of fixed shell 201. In such structure, interior surface 210 of movable shell 202 can be facing the lateral plane, while interior surface 210 of fixed shell 201 can be facing the medial plane. Similar with PACS system 100 with lateral opening 701, the medial half of fixed shell 201 can attach to vertical support proximal end 103. Furthermore, fixed shell proximal end 204 and movable shell proximal end 206 can also be articulated with hinges 211, each hinge 211 proximally on the anterior section and posterior section of PACS system 100. In this embodiment however, fixed shell 201 can support the lateral portion of the lower leg while movable shell 202 can support the medial portion of the lower leg.

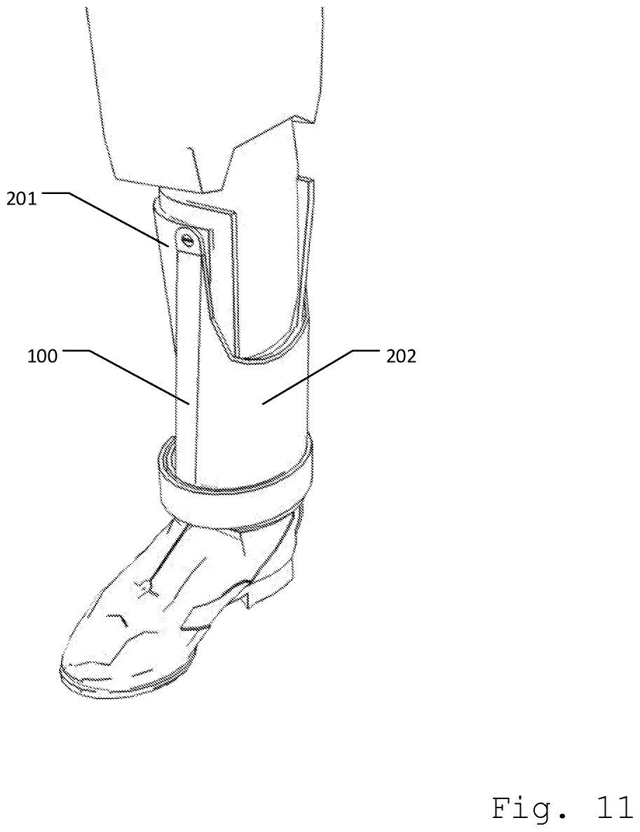

FIG. 11 illustrates PACS system 100 with medial opening 1001 worn on the leg. In this embodiment, the user can put PACS system 100 into open position 100a by swinging movable shell 202 medially away from fixed shell 201. This can create an enlarge medial opening 1001 that can facilitate the insertion of the lower leg within PACS system 100. Once the lower leg is in place, the user can put PACS system 100 into close position 100b by pulling movable shell 202 towards the medial portion of the lower leg. Then, fastening device 203 can be used to wrap PACS system 100 with the lower leg. This system can apply a laterally directed force on the distal tibia and will stabilize the ankle by shifting the ground reaction force medial to the ankle joint. This can be used in treating valgus instabilities such as charcot foot, pes plano valgus etc.

For purposes of these disclosures, donning and doffing embodiments of PACS system 100 can be done with one hand. The user, such as a patient can hold PACS system 100 with one hand and insert the affected (ipsilateral) foot into leg access 200 of PACS system 100. PACS system 100 can be unlike the conventional clamshell design, which can require the use of both hands to fasten the anterior and posterior shells together. Furthermore, users that are suffering from edema can use PACS system 100. The design of PACS system 100 can allow the openings on the proximal section and distal section of the clamshell to allow the circulation of fluid into the legs even while user is wearing PACS system 100. Additionally, when more pressure is needed to correct the deformity of the legs, the user can push movable shell 202 towards fixed shell 201 to create the needed pressure, and then fastening device 203 can be used to stabilize the position of PACS system 100 on the legs. Unlike conventional method, with PACS system 100 additional strap may not be needed to add pressure on the legs thereby preventing pressure sores on the user's leg. Furthermore, fastening device 203 of PACS system 100 does not go around a dorsi flexor on the leg. As such, fastening device 203 does not obstruct the contraction of the Tibialis Anterior, which can then allow free movement of dorsiflexion.

Various changes in the details of the illustrated operational methods are possible without departing from the scope of the following claims. Some embodiments may combine the activities described herein as being separate steps. Similarly, one or more of the described steps may be omitted, depending upon the specific operational environment the method is being implemented in. It is to be understood that the above description is intended to be illustrative, and not restrictive. For example, the above-described embodiments may be used in combination with each other. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.”

1. A proximal articulation clamshell (PACS) system comprising
a fixed shell comprising a fixed shell proximal end and a fixed shell distal end, said fixed shell form-fit with a portion of a lower leg, said fixed shell extending approximately half way around the lower leg;
a movable shell comprising a movable shell proximal end and a movable shell distal end, said moveable shell form-fit with a second portion of the lower leg, said movable shell extending approximately half-way around the lower leg, said movable shell proximal end connected with said fixed shell proximal end wherein said movable shell articulates in relation to said fixed shell, said movable shell capable of being in
an open position wherein said fixed shell and said movable shell form an opening that is capable of receiving the lower leg; and
a closed position wherein said fixed shell and said movable shell together extend substantially around the lower leg; and
a fastening device at the moveable shell distal end that secures said movable shell in said closed position.

2. The PACS of claim 2 further comprising a vertical support, said vertical support comprising a vertical support proximal end and a vertical support distal end, said vertical support proximal end mounted to said fixed shell.

3. The PACS system of claim 1 further wherein said opening is an anterior opening, wherein a pair of hinges are on medial and lateral sides proximally such that said fixed shell is positionable at a posterior section of the lower leg and said movable shell is positionable at an anterior section of the lower leg.

4. The PACS system of claim 3 wherein swinging said movable shell forward and away from said fixed shell in the direction of a sagittal plane enlarges said anterior opening.

5. The PACS system of claim 3 wherein said fixed shell is shorter than said movable shell such that said posterior portion of said fixed shell provides less contact to the surface of the lower leg.

6. The PACS system of claim 1 further wherein said opening is a lateral opening, wherein a pair of hinges connects said fixed shell to said movable shell on an anterior and a posterior portion of said PACS, further wherein said fixed shell is positionable at a medial section of the lower leg and said movable shell is positionable at a lateral section of the lower leg.

7. The PACS system of claim 6 wherein swinging said movable shell laterally away from said fixed shell enlarges said lateral opening.

8. The PACS system of claim 6 wherein said fixed shell and said movable shell are substantially the same length.

9. The PACS system of claim 1 further wherein said opening is a medial opening, wherein a pair of hinges connect said fixed shell and said movable shell on an anterior and a posterior portion of said PACS, further wherein said fixed shell is positionable at a lateral section of the lower leg and said movable shell is positionable at a medial section of the lower leg.

10. The PACS system of claim 9 wherein swinging said movable shell medially away from said fixed shell enlarges said medial opening.

11. The PACS system of claim 9 wherein said fixed shell and said movable shell are substantially the same length.

12. The PACS system of claim 1 wherein said fastening device is a strap, one end of said strap attached at said movable shell distal end while the other end of said strap wraps around said fixed shell and said movable shell.

13. The PACS system of claim 16 wherein said strap comprises a hook and loop fastener.

14. A method of wearing and removing an improved proximal articulation clamshell (PACS) system comprising the steps of
putting a PACS system into an open position, said PACS comprising
a fixed shell comprising a fixed shell proximal end and a fixed shell distal end, said fixed shell form-fit with a portion of a lower leg, said fixed shell extending approximately half way around the lower leg;
a movable shell comprising a movable shell proximal end and a movable shell distal end, said moveable shell form-fit with a second portion of the lower leg, said movable shell extending approximately half-way around the lower leg, said movable shell proximal end connected with said fixed shell proximal end wherein said movable shell articulates in relation to said fixed shell, said movable shell capable of being in
said open position wherein said fixed shell and said movable shell form an opening that is capable of receiving the lower leg; and
a closed position wherein said fixed shell and said movable shell together extend substantially around the lower leg; and
a fastening device at the moveable shell distal end that secures said movable shell in said closed position;
placing said lower leg within PACS system;
putting a PACS system into said closed position;
securing said PACS system into said closed position using said fastening device.

15. The method of claim 14 wherein said opening is an anterior opening, further wherein said PACS system treats equinus foot.

16. The method of claim 14 wherein said opening is a lateral opening, further wherein said PACS system treats varus foot.

17. The method of claim 14 wherein said opening is a medial opening further wherein said PACS system treats valgus foot.
